(19) United States
(12) Patent Application Publication
(10) Pub. No.: US 2021/0178136 A1
(43) Pub. Date: Jun. 17, 2021

(54) IMPLANTABLE MEDICAL DEVICE FOR LOCOREGIONAL INJECTION

(71) Applicants: UNIVERSITÉ D'ANGERS, Angers (FR); CENTRE NATIONAL DE LA RECHERCHE SCIENTIFIQUE, Paris (FR); Inserm (Institut National De La Sante Et De La Recerche Medicale), Paris Cedex (FR)

(72) Inventors: Brice CALVIGNAC, Trelaze (FR); Jean-Christophe GIMEL, Angers (FR); Laurent LEMAIRE, Beaucouze (FR); Florence FRANCONI, Beaucouze (FR)

(21) Appl. No.: 16/319,686

(22) PCT Filed: Jul. 21, 2017
(86) PCT No.: PCT/FR2017/052013

§ 371 Date: Jan. 22, 2019

(30) Foreign Application Priority Data
Jul. 21, 2016 (FR) 1656954

(51) Int. Cl. A61M 37/00

(52) U.S. Cl. CPC A61M 37/0015; A61M 2210/12; A61M 2037/003

(57) ABSTRACT

Disclosed is an implantable medical device for locoregional injection and/or sampling in the lumen of a blood vessel or in a parenchyma, including a microfluidic chip and a cover. The microfluidic chip includes at least one microfluidic channel extending from a first face of the microfluidic chip to a second face of the microfluidic chip. The cover includes at least two hollow micro-needles protruding from the cover. The cover is fixed to the second surface of the microfluidic chip so the microfluidic channel is in fluid connection with the at least two hollow micro-needles. The length of the hollow micro-needles projecting from the cover is configured such that when the cover is implanted on the outer wall of a blood vessel or on a parenchyma, the end of the hollow micro-needles penetrates into the lumen of the blood vessel or into the parenchyma.

### FIELD OF THE INVENTION

This invention relates to the field of medical devices, more specifically implantable microfluidic medical devices for the loco-regional injection of therapeutic molecules. This invention in particular relates to an implantable medical device comprising a microfluidic chip comprising at least one microfluidic channel and a cover comprising at least two hollow micro-needles in fluid connection with the at least one microfluidic channel

### PRIOR ART

The administration of therapeutic molecules is a key aspect of treating a disease. However, the crossing of biological barriers and the locoregional administration of therapeutic molecules for treating diseases affecting deep and hard-to-reach areas of the body are additional obstacles that must be taken into account. More specifically, the systemic route is not always suitable for all treatments. In particular, the systemic route, unlike local administration, leads to the dilution of the therapeutic molecules in the bloodstream. Moreover, this mode of administration can be limiting in terms of the effective dose, degradation and side effects of the molecules administered, such as for example siRNAs, proteins or antibodies.

Targeting a specific organ by the route of administration of the treatment makes it possible to increase therapeutic efficacy while limiting side effects. This is the case, for example, of the intra-parenchymal or intra-arterial route of administration.

Intra-arterial administration upstream of the target is sometimes employed, such as in the case of, for example, chemotherapeutic agents for treating liver cancer. When a tumour is growing in the liver, it receives almost all of its blood supply from the hepatic artery. Intra-arterial chemotherapy thus makes it possible to deliver chemotherapy doses directly to the tumour site that are significantly higher than the doses delivered by a systemic route, by avoiding dilution of the molecules.

This method is currently implemented by means of a catheter inserted into the groin and guided to the artery that irrigates the tumour. The results obtained with this route of administration result in fewer side effects than with standard chemotherapy, however can lead to many complications, such as infection or thrombosis of the artery and/or catheter, which occurs in 30% of cases (S. Bachetti et al., Intra-arterial hepatic chemotherapy for unresectable colorectal liver metastases: a review of medical devices complications in 3172 patients, Medical Devices: Evidence and Research, vol. 2, p31-40, 2009).

In recent years, efforts have been made with devices for locoregional drug administration.

In particular, Gliadel® implants implanted in the cavity formed after the resection of a brain tumour are known. However, these implants do not allow for a controlled and continuous injection, nor for a change in the injected substance (Andrew J. Sawyer et al.,Neiv methods for direct delivery of chemotherapy for treating brain tumors. Yale J Riol Med 2006; 79:141-152).

Patent documents U.S. Pat. Nos. 6,123,861 and 7,918,842 disclose an implantable medical device for administering drugs in a controlled manner through the presence of reservoirs containing the therapeutic molecules. However, this device does not allow for continuous and long-term administration since it must be replaced when the reservoirs are empty.

International patent applications WO 2009/053919 and WO 2011/006699 disclose devices for an intradermal or transdermal injection. Although these devices allow for the continuous administration of therapeutic molecules, these molecules are delivered by the systemic route, with the drawbacks that this route of administration entails.

There is therefore a need for a medical device allowing for the targeted, controlled and continuous administration of drugs, in order to improve the efficacy of the treatments as well as the quality of life of the patients.

This invention thus relates to an implantable microfluidic medical device that is minimally invasive and biocompatible, allowing for the locoregional, controlled and continuous administration of therapies.

### SUMMARY

This invention relates to an implantable medical device for locoregional injection and/or sampling in the lumen of a blood vessel or in a parenchyma comprising a microfluidic chip and a cover, wherein the microfluidic chip comprises at least one microfluidic channel extending from a first face of the microfluidic chip to a second face of the microfluidic chip. The cover comprises at least two hollow micro-needles protruding from the cover, the cover is fixed to the second face of the microfluidic chip such that the at least one microfluidic channel is in fluid connection with the at least two hollow micro-needles, and the length of the at least two hollow micro-needles protruding from the cover is configured such that when the cover is implanted on the outside wall of the blood vessel or on the parenchyma, the end of the at least two hollow micro-needles penetrates the lumen of the blood vessel or the parenchyma.

In one embodiment, the material of the chip and the material of the cover are capable of conforming to the external surface of the blood vessel or parenchyma so as to adapt to the shape of the blood vessel or parenchyma.

In one embodiment, the material of the chip and the material of the cover are plastically conformable, preferably plastically conformable to the external surface of the blood vessel or parenchyma so as to adapt to the shape of the blood vessel or parenchyma.

In one embodiment, the microfluidic chip and the cover are preformed in the shape of a curvature.

In one embodiment, the chip and the cover are preformed in the shape of the blood vessel or parenchyma.

In one embodiment, the first face of the microfluidic chip and the second face of the microfluidic chip are separate. In one embodiment, the microfluidic chip comprises a top face, a bottom face and side faces and the first face is a side face and the second face is a top or bottom face.

In another embodiment, the microfluidic chip comprises a top face, a bottom face and side faces; and the first face is a top face and the second face is a bottom face.

In one embodiment, said cover comprises at least 5, 10, 20, 50 or 100 hollow micro-needles, whereby each hollow micro-needle is in fluid connection with at least one microfluidic channel.

In one embodiment, at least one microfluidic channel can be connected to a primary fluid injection or sampling path.

In one embodiment, the primary path is a catheter.

In one embodiment, the microfluidic chip comprises at least 2 microfluidic channels.

In one embodiment, the microfluidic chip comprises at least two microfluidic circuits.

In one embodiment, each microfluidic circuit can be connected to a separate primary path.

In one embodiment, at least one microfluidic circuit is used for injecting fluid and at least one second microfluidic circuit is used for sampling fluid.

In one embodiment, this invention relates to an implantable medical device for locoregional injection and/or sampling in the lumen of a blood vessel or in a parenchyma excluding blood vessels, vascular smooth muscle cells and endothelial cells.

This invention further relates to a cytotoxic antibiotic, an antimicrotubule agent, a protein kinase inhibitor, a platinum-based agent, an antimetabolite, a siRNA, or a radiosensitiser for treating a liver tumour or liver metastases, which is administered to a patient in need thereof by means of the implantable medical device for locoregional injection according to this invention.

This invention relates to an alkylating agent, a protein kinase inhibitor, a platinum-based agent, an EGFR inhibitor, a VEGF inhibitor, a topoisomerase inhibitor, an antimetabolite, a siRNA or a radiosensitiser for treating a brain tumour, which is administered to a patient in need thereof by means of the implantable medical device for locoregional injection according to this invention.

This invention relates to a cytotoxic antibiotic, an antimicrotubule agent, a platinum-based agent, an antimetabolite, a siRNA or a radiosensitiser for treating a pancreatic tumour, which is administered to a patient in need thereof via the implantable medical device for locoregional injection according to this invention.

### Definitions

In this invention, the terms below shall be understood as follows:
“About”: placed in front of a number, means plus or minus 10% of the nominal value of this number, preferably plus or minus 5% of the nominal value of this number.
“Microfluidic chip”: relates to a substrate in which at least one microfluidic channel is etched, moulded or printed.
“Microfluidic channel”: relates to a channel whose characteristic dimension allows for the flow of fluids such as liquids or gases. The microfluidic channel can be delimited by a bottom wall and two opposite side walls; the distance between the opposite side walls is the characteristic distance. The characteristic distance of the channel lies in the range of about 100 micrometres to about 2,000 micrometres, preferably of about 150 micrometres to about 1,000 micrometres, even more preferably to about 500 micrometres. The microfluidic channel can be a cylindrical channel, the diameter whereof is the characteristic distance.
“Microfluidic circuit”: relates to a microfluidic channel or a set of microfluidic channels in fluid connection inside the substrate.
“Cover”: relates to an element at least partially covering the microfluidic chip. The cover ensures the connection between the at least two hollow micro-needles and the microfluidic chip. When the microfluidic channel is delimited by a bottom wall and two side walls, the cover forms the top wall of the microfluidic channel.
“Primary path”: relates to a fluid connection outside the microfluidic device between an injection or sampling device and the microfluidic chip, in particular between an injection or sampling device and the at least one channel of the microfluidic chip. This primary path allows for the injection or sampling of fluid.
“Secondary path”: relates to a fluid connection inside the microfluidic device from the microfluidic chip to the end of the at least two hollow micro-needles, in particular between the end of the at least one channel of the microfluidic chip opening out onto the edge of the substrate and the end of the at least two hollow micro-needles. This secondary path allows for the injection or sampling of fluid.
“Hollow micro-needle”: relates to a hollow needle, the external diameter whereof lies in the range of about 10 micrometres to about 500 micrometres. It constitutes, with the at least one microfluidic channel, the secondary path for a fluid.
“Subject”: refers to an animal, preferably a mammal, preferably a human.

As understood herein, a subject can be a patient, i.e. a person receiving medical attention, waiting to undergo, undergoing or having undergone medical treatment, and/or being monitored as regards the evolution of a disease.
“Treatment” or “to treat”: mean to prevent, reduce or relieve at least one symptom or negative effect of a disease, disorder or condition associated with the insufficient or failed functioning of an organ or tissue.
“Parenchyma”: means the tissues of an organ that perform the specific functions of that organ and which usually comprises the essential and major bulk of that organ. The parenchyma is distinguished from the stroma which includes, for example, the connective tissues, blood vessels, nerves and ducts (for example the bile ducts) that are not part of the parenchyma.

### DETAILED DESCRIPTION

This invention relates to an implantable medical device (1) for locoregional injection and/or sampling of fluid comprising a microfluidic chip (13) comprising at least one microfluidic channel (121) and a cover (14) comprising at least two hollow micro-needles (11) in fluid connection with the at least one microfluidic channel (121). FIG. 1 shows one embodiment of such an implantable medical device for locoregional injection and/or sampling.

As shown in FIG. 1, the implantable medical device for locoregional injection (11) comprises a microfluidic chip (13), a cover (14) and at least two hollow micro-needles (11). The microfluidic chip comprises at least one microfluidic channel (121) forming the secondary path (12) with the hollow micro-needles. The device according to the invention can further comprise an injection or sampling device (2) connected to the microfluidic chip (13) by a primary path (3).

According to one embodiment, the microfluidic chip (13) comprises at least one substrate made of one or more biocompatible materials chosen from the group consisting of glass, ceramics, metals and metal alloys, silicon, silicone or polymers such as a polydimethylsiloxane (PDMS), a poly(diol-co-citrate) (POC), a cyclic olefin copolymer (COC), parylene, a polyester, a polycarbonate, a polyurethane, a polyamide, polyethylene terephthalate (PET), a polymethylmethacrylate (PMMA), a SU-8 resin, a polylactic acid (PLA), a polyglycolic acid (PGA), a poly(lactic-co-glycolic acid) (PLGA) or a polycaprolactone (PCL). According to one embodiment, the microfluidic chip (13) comprises at least one substrate made of one or more biodegradable materials.

According to one embodiment, the microfluidic chip (13) has a length (L) that lies in the range 1 to 200 millimetres (mm), preferably in the range 2 to 100 mm, preferably the microfluidic chip (13) has a length of about 20 mm

According to one embodiment of the invention, the microfluidic chip (13) has a width (1) that lies in the range 1 to 200 millimetres (mm), preferably in the range 2 to 100 mm, preferably the microfluidic chip (13) has a width of about 20 mm

According to one embodiment of the invention, the microfluidic chip (13) has a surface area that lies in the range 4 to 40,000 mm², preferably in the range 20 to 10,000 mm², preferably the microfluidic chip (13) has a surface area of about 400 mm².

According to one embodiment, the microfluidic chip (13) has the shape of a quadrilateral, preferably a rectangle. According to an alternative embodiment, the microfluidic chip (13) has a U-shape. This last embodiment is particularly advantageous for partially surrounding an object, such as a blood vessel (5), for example in the case of an arterial bypass.

According to one embodiment, the microfluidic chip (13), in particular the material of the substrate, is capable of conforming to the surface on which said implantable medical device (1) is implanted. Preferably, the microfluidic chip (13) is capable of plastically conforming to the surface on which it is implanted.

According to an alternative embodiment, the microfluidic chip (13), in particular the substrate, is preformed according to the configuration of the surface on which said implantable medical device (1) is implanted.

According to one embodiment, the microfluidic chip (13) and the cover (14) are preformed in the shape of a curvature.

Thus, as shown in FIG. 3B in the case of implantation on the external surface of a blood vessel (5), the microfluidic chip (13) is either capable of conforming to the external surface of said blood vessel (5) or preformed in the shape (for example the curvature) of the external surface of said blood vessel (5).

As shown in FIG. 3A, in the case of intraparenchymal implantation (4) for example in an excision cavity, the microfluidic chip (13) is preferably capable of conforming to the surface of the cavity in which it is implanted. Indeed, it is difficult to predict the shape of the excision cavity before the operation and therefore to obtain a preformed microfluidic chip (13).

The microfluidic chip (13) comprises a substrate comprising at least one microfluidic channel (121). The substrate comprises a top face, a bottom face and side faces. Said at least one microfluidic channel (121) extends from a first face of the substrate to a second face of the substrate. Said first face of the substrate can be a bottom, top or side face. The opening of the at least one microfluidic channel (121) onto the first face of the substrate can be connected to a primary path (3). Said second face of the substrate can be a bottom, top or side face. In one embodiment, the first face of the microfluidic chip (13) and the second face of the microfluidic chip (13) are separate. According to one embodiment, the first face is a top face and the second face is a bottom face. According to one embodiment, the first face is a side face and the second face is a top or bottom face. In this latter embodiment, a primary path (3) can be connected to the microfluidic channel (121) on a side face of the chip, so as to minimise the overall dimensions of the implantable device. In particular, when the device is implanted on a blood vessel (5), the primary path (3) can be connected to the chip by at least partially running alongside the blood vessel (5).

According to one embodiment, said at least one microfluidic channel (121) extends from the centre of the first face of the substrate.

According to one embodiment of the invention, the substrate comprises 2, 3, 4, 5, 6, 7, 8, 9, 10, 12, 15, 20, 40, 50 or 100 microfluidic channels (121). According to one embodiment of the invention, the substrate comprises at least 2, 3, 4, 5, 6, 7, 8, 9, 10, 12, 15, 20, 40, 50 or 100 microfluidic channels (121).

According to one embodiment wherein the substrate comprises at least two microfluidic channels (121), each microfluidic channel (121) forms a different microfluidic circuit.

According to one embodiment wherein the substrate comprises at least two microfluidic channels (121), the set of microfluidic channels (121) forms a single microfluidic circuit. According to one embodiment wherein the substrate comprises a single microfluidic channel (121), this channel forms a microfluidic circuit.

According to one embodiment wherein the substrate comprises at least two microfluidic channels (121), the microfluidic channels (121) are combined so as to form a plurality of microfluidic circuits.

According to one embodiment, a separate primary path (3) feeds each microfluidic circuit. According to one embodiment, the same primary path (3) feeds a plurality of microfluidic circuits. According to one embodiment, a plurality of primary paths feed the same microfluidic circuit. This latter embodiment allows for the simultaneous injection of different fluids into a microfluidic circuit.

According to one embodiment, the primary path (3), or the plurality of primary paths, can be any system for injecting a fluid, preferably a liquid, into the channels of the microfluidic chip (13); preferably the primary path (3) is a catheter. According to one embodiment, a primary path (3) can sequentially inject different fluids into the microfluidic chip (13).

According to one embodiment wherein the substrate comprises at least two microfluidic channels (121), the substrate comprises at least two microfluidic circuits. In this embodiment, the device can comprise two primary paths, the first primary path (3) being connected to the first microfluidic circuit and the second primary path (3) being connected to the second microfluidic circuit. This embodiment allows for the injection of different fluids (for example different therapeutic molecules) into separate microfluidic circuits. This embodiment also allows for the use of a first microfluidic circuit for fluid injection and the use of a second microfluidic circuit for fluid sampling.

According to one embodiment wherein the substrate comprises at least three microfluidic channels (121), the substrate comprises at least three microfluidic circuits. In this embodiment, the device can comprise three primary paths, each primary path (3) being connected to a microfluidic circuit. This embodiment allows, for example, a first microfluidic circuit to be used for injecting an active ingredient, a second microfluidic circuit to be used for injecting an eluent, such as a physiological fluid, and a third microfluidic circuit to be used for sampling a fluid, in particular for sampling interstitial fluid after elution.

A person skilled in the art will easily be able to adapt this embodiment in order to have as many primary paths and microfluidic circuits as required.

The cover (14) according to the invention makes it possible to secure the at least two hollow micro-needles (11) to the microfluidic chip (13). The cover (14) comprises the at least two hollow micro-needles (11). According to one embodiment, the cover (14) secures at least two micro-needles (11) to the microfluidic chip (13).

According to one embodiment, the cover (14) is fixed onto the second face of the microfluidic chip (13) (i.e. the face onto which the at least one microfluidic channel (121) opens out). Thus, the cover (14) preferably has the same shape as the microfluidic chip (13).

According to one embodiment, the cover (14) is made of one or more biocompatible materials chosen from the group consisting of glass, ceramics, metals and metal alloys, silicon, silicone or polymers such as a polydimethylsiloxane (PDMS), a poly(diol-co-citrate) (POC), a cyclic olefin copolymer (COC), parylene, a polyester, a polycarbonate, a polyurethane, a polyamide, polyethylene terephthalate (PET), a polymethylmethacrylate (PMMA), a SU-8 resin, a polylactic acid (PLA), a polyglycolic acid (PGA), a poly(lactic-co-glycolic acid) (PLGA) or a polycaprolactone (PCL). According to one embodiment, the cover (14) is made of one or more biodegradable materials.

According to one embodiment, as shown in FIG. 2A, the cover (14) and the hollow micro-needles (11) form two separate elements. In this embodiment, the at least two hollow micro-needles (11) are fixed onto the cover (14), which includes perforations for connecting the microfluidic channel (121) of the microfluidic chip (13) to the hollow micro-needles (11).

According to one embodiment, as shown in FIG. 2B, the cover (14) and the hollow micro-needles (11) form two separate elements. In this embodiment, the cover (14) comprises at least two openings designed to receive the at least two micro-needles. According to one embodiment, in order to simplify the assembly of the hollow micro-needles (11) with the cover (14), and as shown in FIG. 2B, the cover (14) comprises a plurality of recesses designed to receive the base of the hollow micro-needles (11).

According to one embodiment, as shown in FIG. 2C, the cover (14) and the at least two hollow micro-needles (11) form one piece. In this embodiment, in order to stiffen the hollow micro-needles (11), said hollow micro-needles (11) can optionally be coated in a metal deposit.

According to one embodiment, the cover (14) is fixed by anchoring on the second face of the microfluidic chip (13), such that the hollow micro-needles (11) are in fluid connection with the at least one microfluidic channel (121).

According to one embodiment, as shown in FIG. 2D, the cover and the microfluidic chip are made in one piece, for example by 3D stereolithography.

According to one embodiment, the material of the cover (14) is identical to the material of the microfluidic chip (13).

According to one embodiment, the material of the cover (14) is capable of conforming to the surface on which said implantable medical device (1) is implanted. Preferably, the cover (14) is plastically conformable to the surface on which the implantable medical device (1) is implanted so as to maximise the contact area between the cover (14) and the targeted tissue and/or organ.

According to an alternative embodiment, the cover (14) is preformed according to the configuration of the targeted surface on which the implantable medical device (1) is implanted.

According to one embodiment, the medical device (1) according to the invention comprises at least two hollow micro-needles (11), preferably at least 3, 4, 5, 6, 7, 8, 9, 10, 12, 15, 20, 40, 50, 100, 200, 300, 400, 500 or 1,000 hollow micro-needles (11). According to one embodiment, the number of hollow micro-needles (11) is identical to the number of microfluidic channels (121) of the microfluidic chip (13). The presence of multiple hollow micro-needles (11) guarantees the durability of the injection in the event that some thereof become blocked. The presence of multiple hollow micro-needles (11) also makes it possible to increase the injection flow rate.

According to one embodiment, each hollow micro-needle (11) of the invention is connected to at least one microfluidic channel (121). According to one embodiment, each hollow micro-needle (11) of the invention is connected to a single microfluidic channel (121). According to another embodiment, each hollow micro-needle (11) of the invention is connected to more than one microfluidic channel (121). In another embodiment, each microfluidic channel (121) is connected to more than one hollow micro-needle (11).

According to one embodiment, the hollow micro-needles (11) of the invention are rigid. The term “rigid” is understood to mean that the hollow micro-needles (11) of the invention can penetrate the wall of a parenchyma (4) or of blood vessels, such as arteries or veins, without becoming deformed or clogged and without breaking.

According to one embodiment, the hollow micro-needles (11) are made of one or more biocompatible materials chosen from the group consisting of glass, ceramics, metals and metal alloys, silicon, silicone or polymers such as a polydimethylsiloxane (PDMS), a poly(diol-co-citrate) (POC), a cyclic olefin copolymer (COC), parylene, a polyester, a polycarbonate, a polyurethane, a polyamide, polyethylene terephthalate (PET), a polymethylmethacrylate (PMMA), a SU-8 resin, a polylactic acid (PLA), a polyglycolic acid (PGA), a poly(lactic-co-glycolic acid) (PLGA) or a polycaprolactone (PCL). In one embodiment, the hollow needles are made of one or more biodegradable materials.

According to one embodiment, the external diameter of the hollow micro-needles (11) of the invention lies in the range 10 to 500 micrometres, preferably in the range 100 to 350 micrometres or in the range 100 to 300 micrometres.

According to one embodiment, the internal diameter of the hollow micro-needles (11) of the invention, i.e. the diameter of the lumen of the micro-needles, lies in the range 1 to 450 micrometres, preferably in the range 50 to 200 micrometres.

According to one embodiment, the hollow micro-needles (11) of the invention have a size, i.e. the distance between the base and the tip of the micro-needles, that lies in the range 100 to 10,000 micrometres, preferably in the range 200 to 2,000 micrometres.

According to one embodiment, the hollow micro-needles (11) of the invention have a size that is greater than 100, 200, 300, 400, 500, 600, 700, 800, 900 or greater than 1,000 micrometres.

According to one embodiment, the hollow micro-needles (11) of the invention have an external diameter and a size that are determined such that the tip of the micro-needle penetrates the lumen of a blood vessel.

According to one embodiment, the hollow micro-needles (11) of the invention have a size that is greater than the thickness of the wall of the blood vessel and that is less than the sum of the thickness of the wall and the diameter of the lumen of the blood vessel.

The upper part, or tip, of the hollow micro-needles (11) of the invention corresponds to the part that penetrates a parenchyma (4) or passes through a blood vessel wall. Conversely, the lower part, or base, of the hollow micro-needles (11) of the invention corresponds to the part connected to at least one microfluidic channel (121) of the microfluidic chip (13) as described hereinabove.

As described above, the cover (14) comprises the at least two hollow micro-needles (11); thus, the hollow micro-needles (11) are located on a single face of the implantable medical device (1).

According to one embodiment, the hollow micro-needles (11) are evenly distributed over the cover (14). According to one embodiment, the hollow micro-needles (11) are distributed in a geometric pattern.

According to one embodiment wherein the substrate comprises at least two microfluidic circuits, the hollow micro-needles (11) in fluid connection with the first microfluidic circuit are grouped together and the hollow micro-needles (11) in fluid connection with the second microfluidic circuit are grouped together, thus forming two clusters of hollow micro-needles (11) on the cover (14).

According to one embodiment wherein the substrate comprises at least two microfluidic circuits, the hollow micro-needles (11) in fluid connection with the first microfluidic circuit are situated at the periphery of the cover (14), whereas the hollow micro-needles (11) in fluid connection with the second microfluidic circuit are situated in the centre of the cover (14).

According to one embodiment, the tip of the hollow micro-needles (11) of the invention is bevelled in order to facilitate penetration of a parenchyma (4) or of a blood vessel wall. According to one embodiment, the tip of the hollow micro-needles (11) is flat. According to one embodiment, the tip of the hollow micro-needles (11) is conical and closed at the end thereof. In this latter embodiment, the hollow micro-needles (11) comprise radial openings.

According to one embodiment, the hollow micro-needles (11) are open at the end of the tip thereof. According to an alternative embodiment, the hollow micro-needles (11) are closed at the end of the tip thereof and comprise a radial opening. According to an alternative embodiment, the hollow micro-needles (11) are closed at the end of the tip thereof and comprise a plurality of radial openings. According to an alternative embodiment, the hollow micro-needles (11) are open at the end of the tip thereof and comprise a plurality of radial openings.

According to one embodiment, the medical device (1) according to the invention is implanted on a tissue, such as the wall of a parenchyma (4), or the wall of a blood vessel (5), preferably an artery. Thus, the device according to the invention allows therapeutic molecules to be administered in a locoregional manner

According to one embodiment, the medical device (1) of the invention is implanted near the organ and/or the tissue to be treated. According to one embodiment, the medical device (1) of the invention is implanted in a precise manner, for example according to stereotaxic coordinates.

Implantation of such a device on a blood vessel (5) in the upstream vicinity of the organ and/or the tissue to be treated prevents the risk of thrombosis linked to the insertion of a catheter into the blood vessel (5). Moreover, in-situ implantation reduces the quantity of therapeutic molecules required for the treatment compared to a systemic route of administration for example. This device also reduces the side effects resulting from systemic administration since only the organ targeted by the treatment is in contact with the therapeutic doses of therapeutic molecules. The device according to the invention further allows for locoregional treatment of diseases affecting the organs and/or deep tissues of the body. Moreover, this device avoids the blood-brain barrier by the in-situ implantation thereof in the brain.

According to one embodiment, the medical device (1) of the invention is implanted on the hepatic artery, on the gastroduodenal artery, or on a branch of these arteries for the administration of therapeutic molecules respectively in the lumen of the hepatic artery, gastroduodenal artery, or a branch of these arteries supplying the liver.

According to another embodiment, the medical device (1) of the invention is implanted on the renal artery for the administration of therapeutic molecules in the lumen of the renal artery supplying the kidneys.

According to another embodiment, the medical device (1) of the invention is implanted on a pulmonary artery for the administration of therapeutic molecules in the lumen of the pulmonary artery supplying the lungs.

According to another embodiment, the medical device (1) of the invention is implanted on the coeliac trunk, the gastroduodenal artery or the splenic artery for the administration of therapeutic molecules respectively in the lumen of the coeliac trunk, gastroduodenal artery or splenic artery supplying the pancreas.

According to another embodiment, the medical device (1) of the invention is implanted on a cerebral artery (anterior, middle or posterior) for the administration of therapeutic molecules in the lumen of a cerebral artery supplying different areas of the brain.

According to another embodiment, the medical device (1) of the invention is implanted in an excision cavity, preferably an excision cavity in the cerebral region.

According to one embodiment of the invention, the medical device (1) of the invention is implanted such that the entire face of the cover (14) opposite that on which the microfluidic chip (13) is fixed is in contact with the targeted tissue.

According to one embodiment, the medical device (1) according to the invention is held in place on the tissue by means of a medical glue, such as an acrylic adhesive, a light-activated adhesive or BioGlue® marketed by Cryolife. According to another embodiment, the device of the invention is held in place by a clip or brace and/or stitches. According to another embodiment, the device of the invention is held in place by stitches. According to one embodiment, the medical device (1) is held in place on the tissue by a combination of the means described above.

According to one embodiment wherein the medical device (1) is implanted on a blood vessel (5), preferably an artery, each of the hollow micro-needles (11) passes through the wall of the vessel and penetrates the lumen of the vessel, preferably in a substantially radial manner. This is possible, as explained above, thanks to the conformable materials of the chip and the cover (14) or thanks to a preformed chip and cover (14).

According to one embodiment wherein the medical device (1) is implanted on a blood vessel (5), as shown in FIG. 3, the distance between the surface of the cover (14) in contact with the vessel and the end of the hollow micro-needles (11) is designed such that the ends of the micro-needles pass through the vessel and penetrate the lumen of the vessel. According to one embodiment, the distance between the surface of the cover (14) in contact with the blood vessel (5) and the end of the hollow micro-needles (11) is designed such that the ends of the hollow micro-needles (11) penetrate the lumen of the blood vessel (5) over a distance that is less than half, preferably less than a quarter of the diameter of the lumen of the vessel, so as not to hinder blood flow.

According to one embodiment, when the device is implanted on a vessel, the distance between the end of the tip of the hollow micro-needles (11) and the inside wall of the blood vessel (5) through which pass said micro-needles is less than or equal to 500 micrometres, preferably less than or equal to 250 micrometres.

In one embodiment, the invention is not a medical device used to administer a treatment directly into the tunica media of the blood vessel.

In one embodiment, the cover (14) and the microfluidic chip (13) do not include an isolated reservoir. In one embodiment, the medical device (1) does not include a plurality of reservoirs where each reservoir is connected to a micro-needle.

The invention further relates to the use of the medical device (1) according to the invention for treating a disease, preferably a disease affecting a deep and/or hard-to-access tissue and/or organ. The device according to the invention is used to treat a disease by the injection of therapeutic molecules either directly into an organ and/or deep tissue, or into the lumen of a blood vessel (5) upstream of the targeted organ and/or deep tissue.

Examples of a deep and/or hard-to-access tissue and/or organ include, but are not limited to, the liver, lungs, pancreas, brain, soft tissue, blood vessels, viscera and bones.

The medical device (1) according to the invention allows for a targeted treatment to be administered, while limiting the side effects on healthy organs and/or tissue. According to one embodiment, the medical device (1) according to the invention can be used for treating a tumour and/or a metastatic growth located in an organ and/or deep tissue.

According to one embodiment, the medical device (1) according to the invention can be used for treating a disease affecting the brain, and for which the administration of therapeutic molecules via the systemic route is prevented by the blood-brain barrier. Examples of diseases affecting the brain include, but are not limited to, brain tumours, neurodegenerative diseases, epilepsy, etc. According to one embodiment, the medical device (1) according to the invention can be used for treating a neurodegenerative disease such as Parkinson's disease.

According to one embodiment, the medical device (1) according to the invention can be used for treating a brain tumour by administering molecules chosen from the group consisting of an alkylating agent such as temozolomide, nimustine or carmustine (BiCNU); a protein kinase inhibitor such as Sorafenib; a platinum-based agent such as cisplatin or carboplatin; an EGFR inhibitor such as erlotinib, cetuximab or gefitinib; a VEGF inhibitor such as vandetanib, bevacizumab (Avastin) or cediranib; a topoisomerase inhibitor such as etoposide; an antimetabolite such as methotrexate; a hyperosmotic agent such as mannitol; a siRNA or a radiosensitiser.

According to one embodiment, the medical device (1) according to the invention can be used for treating a liver tumour or liver metastasis by administering molecules chosen from the group consisting of a cytotoxic antibiotic such as doxorubicin; an antimicrotubule agent such as paclitaxel; a protein kinase inhibitor such as sorafenib or irinotecan; a platinum-based agent such as oxaliplatin or cisplatin; an antimetabolite such as fluorouracil (5-FU), gemcitabine or floxuridine; a siRNA or a radiosensitiser.

According to one embodiment, the medical device (1) according to the invention can be used for treating a pancreatic tumour by administering molecules chosen from the group consisting of a cytotoxic antibiotic such as mitomycin, mitoxantrone, epirubicin or doxorubicin; an antimicrotubule agent such as paclitaxel; a platinum-based agent such as carboplatin; an antimetabolite such as fluorouracil (5-FU) or gemcitabine; a siRNA or a radiosensitiser.

According to one embodiment, the medical device (1) according to the invention can be used for treating a sarcoma by administering antitumour agents into the excision cavity.

According to one embodiment, the medical device (1) according to the invention can be used for treating stenosis by administering an antimicrotubule agent such as paclitaxel into the wall of the artery. In this embodiment, the micro-needles are designed not to penetrate the lumen of the artery, only the arterial wall.

According to one embodiment, the therapeutic molecules that can be injected by means of the medical device (1) of the invention include all molecules that can be administered in liquid form. Examples of therapeutic molecules include, but are not limited to, antitumour agents, siRNAs, proteins, stem cells and antibodies.

The medical device (1) of the invention is implanted and connected to a primary path (3) carrying the therapeutic molecules. Thus, the medical device (1) of the invention avoids the need for repeated injections and allows action to be taken quickly in the event of a localised relapse. According to one embodiment, the medical device (1) according to the invention can be used for treating a disease that requires the repetitive and frequent administration of therapeutic agents. In another embodiment, the medical device (1) according to the invention can be used for treating a disease that requires controlled administration, depending on the state of evolution of the disease. In another embodiment, the medical device (1) according to the invention can be used for treating a disease that is likely to recur. In another embodiment, the medical device (1) according to the invention can be used for administering treatment immediately after an operation.

According to one embodiment of the invention, the primary path (3) is used for administering fluid, preferably liquid. According to one embodiment of the invention, the primary path (3) is used for sampling fluid, preferably liquid.

According to one embodiment, the primary path (3) is remotely controlled by an external pump (conventional syringe driver) or an implantable pump.

According to one embodiment, the administration of fluid, preferably liquid, is continuous.

According to another embodiment, the administration of fluid, preferably liquid, is discontinuous. According to one embodiment, the liquid is administered 1, 2, 3 or 4 times a day, or more. According to another embodiment, the liquid is administered 1, 2, 3, 4, 5, 6 or 7 times a week or every 2 weeks. According to another embodiment, the liquid is administered 1, 2, 3, 4, 5, 6 or 7 times a month. According to one embodiment, administration is, for example, continuous over a period of 1 month, then stopped for a period of 1 month, then continuous again for a period of 1 month, and so on. Administration can also be continuous for a period of 6 months, then stopped for a period of 6 months, then continuous again for a period of 6 months, and so on.

The medical device (1) of the invention allows actions to be taken quickly in the event of a relapse. Thus, according to one embodiment of the invention, the administration of liquid can be resumed after a long period of stopped treatment.

According to one embodiment, the administration of liquid is controlled according to the evolution of the disease. The medical device (1) of the invention thus allows treatment to be tailored to suit the individual needs of each patient.

According to one embodiment, the medical device (1) according to the invention can be used for treating a disease requiring the administration of therapeutic agents at sub-toxic doses for the treatment to be effective.

The invention also relates to a therapeutic molecule administered by means of the medical device (1) as described above.

The invention therefore further relates to a substance for treating a disease, characterised in that it is administered to a patient in need thereof by means of the device as described above.

According to one embodiment, the therapeutic molecule is used for treating a disease chosen from the group consisting of a brain tumour, a liver tumour, a liver metastasis, a pancreatic tumour, or arterial stenosis. According to one embodiment, the therapeutic molecule is not used for treating arterial stenosis, hyperplasia, an abnormal growth in vascular smooth muscle cells or for treating endothelial cell damage.

According to one embodiment, the therapeutic molecule used for treating a brain tumour is chosen from the group consisting of an alkylating agent such as temozolomide, nimustine or carmustine (BiCNU); a protein kinase inhibitor such as Sorafenib; a platinum-based agent such as cisplatin or carboplatin; an EGFR inhibitor such as erlotinib, cetuximab or gefitinib; a VEGF inhibitor such as vandetanib, bevacizumab (Avastin) or cediranib; a topoisomerase inhibitor such as etoposide; an antimetabolite such as methotrexate; a hyperosmotic agent such as mannitol; a siRNA or a radiosensitiser.

According to one embodiment, the therapeutic molecule used for treating a liver tumour or liver metastasis is chosen from the group consisting of a cytotoxic antibiotic such as doxorubicin; an antimicrotubule agent such as paclitaxel; a protein kinase inhibitor such as sorafenib or irinotecan; a platinum-based agent such as oxaliplatin or cisplatin; an antimetabolite such as fluorouracil (5-HU), gemcitabine or floxuridine; a siRNA or a radiosensitiser.

According to one embodiment, the therapeutic molecule used for treating a pancreatic tumour is chosen from the group consisting of a cytotoxic antibiotic such as mitomycin, mitoxantrone, epirubicin or doxorubicin; an antimicrotubule agent such as paclitaxel; a platinum-based agent such as carboplatin; an antimetabolite such as fluorouracil (5-FU) or gemcitabine; a siRNA or a radiosensitiser.

According to one embodiment, the therapeutic molecule is an antimicrotubule agent such as paclitaxel for treating stenosis.

According to one embodiment, the use of the medical device (1) of the invention is combined with at least one other treatment. According to one embodiment, the at least one other treatment is intended to treat the same disease as the medical device (1) of the invention. According to another embodiment, the at least one other treatment is intended to treat a disease that is different to that treated by the medical device (1) of the invention.

According to one embodiment, the use of the medical device (1) of the invention is combined with a tumourostatic treatment based on anti-angiogenic molecules. Examples of antitumour molecules include, but are not limited to, alkylating agents, antimetabolites, antitumour antibiotics, topoisomerase inhibitors, microtubule inhibitors, monoclonal antibodies, or protein kinase inhibitors.

Other examples of treatments that can be combined with the use of the medical device (1) of the invention include, but are not limited to, radioembolisation, chemoembolisation, radiosensitisation for external beam radiotherapy, surgery or the oral administration of medication.

According to one embodiment, the subject has already followed another course of treatment before the implantation of the medical device (1) of the invention. According to one embodiment, the subject has undergone surgery prior to the implantation of the medical device (1) of the invention, such as resection surgery. According to one embodiment, the implantation of the medical device (1) of the invention takes place during an operation, such as resection surgery.

In another embodiment, the subject has not yet followed any other course of treatment before the implantation of the medical device (1) of the invention.

### BRIEF DESCRIPTION OF THE FIGURES

FIG. 1 is an exploded view of one embodiment of the implantable medical device for locoregional injection according to this invention.

FIG. 2A is a sectional view of one embodiment of this invention, wherein the cover and the hollow micro-needles form two separate elements. In this embodiment, the micro-needles are positioned on the cover.

FIG. 2B is a sectional view of one embodiment of this invention, wherein the cover and the hollow micro-needles form two separate elements. In this embodiment, the micro-needles pass through the cover.

FIG. 2C is a sectional view of one embodiment of this invention, wherein the cover and the hollow micro-needles are formed in one piece.

FIG. 2D is a sectional view of one embodiment of this invention, wherein the cover, the micro-needles and the microfluidic chip are formed in one piece.

FIG. 3A is a diagram of the implantable medical device for locoregional injection according to one embodiment of this invention during a locoregional injection into a parenchyma.

FIG. 3B is a diagram of the implantable medical device for locoregional injection according to one embodiment of this invention during a locoregional injection into the lumen of a blood vessel.

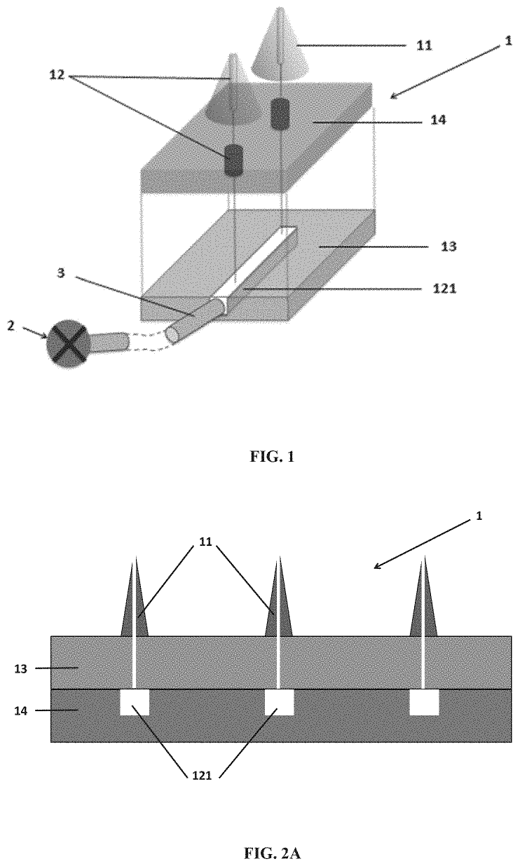

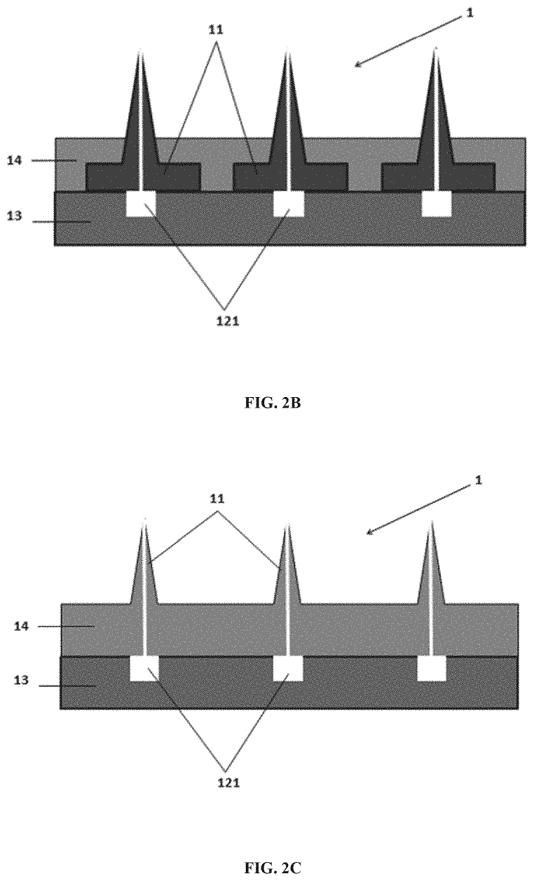

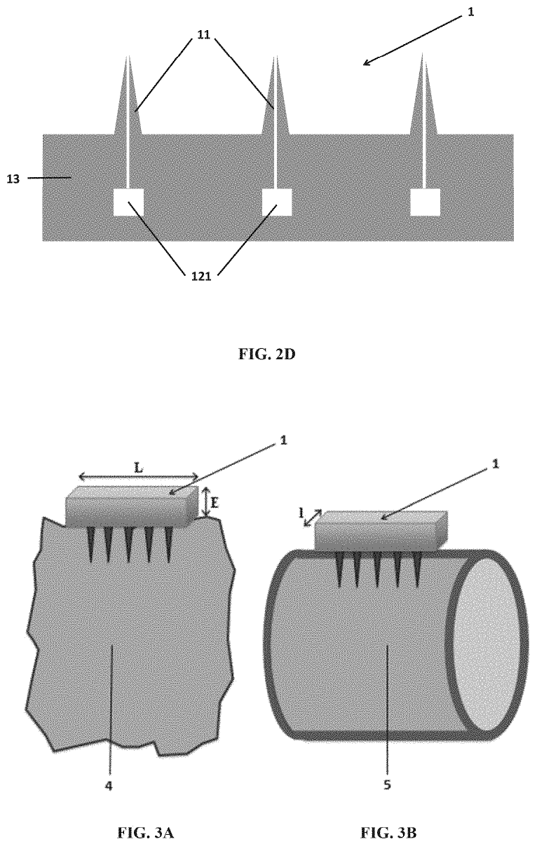

### REFERENCES

1—Implantable medical microfluidic device for locoregional injection

11—Micro-needles

12—Secondary path

121—Microfluidic channel

13—Microfluidic chip

14—Cover

2—Injection/sampling device

3—Primary path

4—Parenchyma

5—Blood vessel

1. Implantable medical device for locoregional injection and/or sampling in the lumen of a blood vessel or in a parenchyma comprising a microfluidic chip and a cover, wherein the microfluidic chip comprises at least one microfluidic channel extending from a first face of the microfluidic chip to a second face of the microfluidic chip, the cover comprises at least two hollow micro-needles protruding from the cover, the cover is fixed to the second face of the microfluidic chip such that the at least one microfluidic channel is in fluid connection with the at least two hollow micro-needles, and the length of the at least two hollow micro-needles protruding from the cover is configured such that when the cover is implanted on the outside wall of the blood vessel or on the parenchyma, the end of the at least two hollow micro-needles penetrates the lumen of the blood vessel or the parenchyma.

2. Device according to claim 1, wherein the material of the microfluidic chip and the material of the cover are plastically conformable.

3. Device according to claim 1, wherein the microfluidic chip and the cover are preformed in the shape of a curvature.

4. Device according to claim 1, wherein the first face of the microfluidic chip and the second face of the microfluidic chip are separate.

5. Device according to claim 4, wherein the microfluidic chip comprises a top face, a bottom face and side faces, and wherein the first face is a side face and the second face is a top or bottom face.

6. Device according to claim 4, wherein the microfluidic chip comprises a top face, a bottom face and side faces, and wherein the first face is a top face and the second face is a bottom face.

7. Device according to claim 1, wherein said cover comprises at least 5, 10, 20, 50 or 100 hollow micro-needles, whereby each hollow micro-needle is in fluid connection with at least one microfluidic channel.

8. Device according to claim 1, wherein said at least one microfluidic channel is connected to a primary fluid injection or sampling path such as a catheter.

9. Device according to claim 1, wherein said microfluidic chip comprises at least 2 microfluidic channels.

10. Device according to claim 9, wherein said microfluidic chip comprises at least two microfluidic circuits.

11. Device according to claim 10, wherein each microfluidic circuit is connected to a separate primary path.

12. Cytotoxic antibiotic, antimicrotubule agent, protein kinase inhibitor, platinum-based agent, antimetabolite, siRNA, or radiosensitiser for treating a liver tumour or liver metastases, administered to a patient in need thereof by means of the device according to claim 1.

13. Alkylating agent, protein kinase inhibitor, platinum-based agent, EGFR inhibitor, VEGF inhibitor, topoisomerase inhibitor, antimetabolite, siRNA or radiosensitiser for treating a brain tumour, administered to a patient in need thereof by means of the device according to claim 1.

14. Cytotoxic antibiotic, antimicrotubule agent, platinum-based agent, antimetabolite, siRNA, or radiosensitiser for treating a pancreatic tumour, administered to a patient in need thereof by means of the device according to claim 1.

15. The device according to claim 1, wherein the material of the microfluidic chip and the material of the cover are plastically conformable to the external surface of the blood vessel or parenchyma so as to adapt to the shape of the blood vessel or parenchyma.

16. Device according to claim 2, wherein the first face of the microfluidic chip and the second face of the microfluidic chip are separate.

17. Device according to claim 3, wherein the first face of the microfluidic chip and the second face of the microfluidic chip are separate.

18. Device according to claim 2, wherein said cover comprises at least 5, 10, 20, 50 or 100 hollow micro-needles, whereby each hollow micro-needle is in fluid connection with at least one microfluidic channel.

19. Device according to claim 3, wherein said cover comprises at least 5, 10, 20, 50 or 100 hollow micro-needles, whereby each hollow micro-needle is in fluid connection with at least one microfluidic channel.

20. Device according to claim 4, wherein said cover comprises at least 5, 10, 20, 50 or 100 hollow micro-needles, whereby each hollow micro-needle is in fluid connection with at least one microfluidic channel.
